(19) United States
(12) Patent Application Publication
(10) Pub. No.: US 2011/0273286 A1
(43) Pub. Date: Nov. 10, 2011

(54) System for Monitoring a Person Wearing Head Gear

(76) Inventor: Frederick H. Sklar, Waxahachie, TX (US)

(21) Appl. No.: 13/095,548

(22) Filed: Apr. 27, 2011

Related U.S. Application Data
(63) Continuation-in-part of application No. 11/208,229, filed on Aug. 19, 2005, now abandoned.
Application No. 11/208,229 is a continuation of application No. 10/620,070, filed on Jul. 14, 2003, now Pat. No. 6,939,316.
(60) Provisional application No. 61/328,831, filed on Apr. 28, 2010.

(51) Int. Cl. A61F 5/00; G08B 23/00

(52) U.S. Cl. 340/539.12; 602/17

(57) ABSTRACT

A cranial orthosis includes a sensor to monitor one or more conditions of an infant wearing the cranial orthosis. The cranial orthosis is preferably contoured to match the curvature of the fronto-temporal, parietal and occipital areas of an infant's cranial vault to provide protection against the acquisition of postural cranial deformities as a result of the infant's sleeping in the supine position. The orthosis is designed to be of universal fit, as determined by the infant's fronto-occipital head circumference (FOC) measurement. The interior dimensions of the orthosis can be enlarged to accommodate growth of the infant's head without requiring replacement. The sensor may detect oxygen saturation, pulse, temperature, or any other measureable condition or combination of conditions. The system includes an alarm that is triggered when a sensed condition crosses a selected threshold level.

### PRIORITY STATEMENT & CROSS-REFERENCE TO RELATED APPLICATIONS

This application claims priority from co-pending U.S. Patent Application Ser. No. 61/328,831, entitled “System for Monitoring a Person Wearing Head Gear” and filed on Apr. 28, 2010 in the name of Frederick H. Sklar. This application is also a continuation-in-part of co-pending U.S. patent application Ser. No. 11/208,229, entitled “Cranial Orthosis for Preventing Positional Plagiocephaly in Infants” and filed on Aug. 19, 2005 in the names of Frederick H. Sklar and Paul C. Hobar; which is a continuation of U.S. patent application Ser. No. 10/620,070, entitled “Cranial Orthosis for Preventing Positional Plagiocephaly in Infants,” filed on Jul. 14, 2003 and issued on Sep. 6, 2005 as U.S. Pat. No. 6,939,316; both of which are hereby incorporated by reference for all purposes.

### FIELD OF THE INVENTION

This invention relates generally to medical devices for preventing and treating cranial deformities in infants, incorporating one or more sensors to monitor the condition of the infant while wearing the appliance.

### DESCRIPTION OF THE RELATED ART

Cranial asymmetry (plagiocephaly) and deformations may occur from various congenital causes including premature closure of the cranial vault and/or skull base sutures (craniosynostosis), syndromal craniofacial dysostosis, intracranial volume disorders such as hydrocephalus, microcephaly or tumor, metabolic bone disorders such as rickets and birth trauma such as depressed skull fractures. Cranial deformity (cranial molding) may also be acquired in an infant as the result of compressive forces imposed by the infant's head weight on the soft, compliant occipital areas while the infant is lying on a sleep surface in the supine position. This condition typically occurs during the first twelve months of development before the cranium is fully expanded and the brain is fully developed.

Generally, plagiocephaly is characterized by unilateral occipital flattening with contralateral occipital bulging, producing a flat spot at the back of the infant's head. The flat spot and bulging make the baby's head appear to be square or box-shaped in profile. As the deformation becomes more severe there is ipsilateral forehead protrusion, contralateral forehead flattening and endocranial skull base rotation with anterior displacement of the ipsilateral ear. If not prevented or corrected during the first twelve months of development, the deformity may become permanent.

The number of infants diagnosed with plagiocephaly increased substantially shortly after the onset of the “Back-to-Sleep” campaign by the American Academy of Pediatrics (AAP) in 1992. In that campaign, the AAP recommended that infants be placed in the supine (lying on the back, face up) sleeping position in an effort to decrease the incidence of sudden infant death syndrome (SIDS), a leading cause of early infantile deaths in the United States at that time. That campaign resulted in a substantial decrease in the incidence of SIDS. However, the incidence of plagiocephaly was observed to increase significantly over the same period. This correlation suggests that positional treatment for SIDS was the probable cause of the increased incidence of infant plagiocephaly. The consensus of craniofacial practitioners is that plagiocephaly may be acquired as a result of cranial postural molding that occurs during SIDS positional treatment. That condition is now referred to as positional plagiocephaly or acquired plagiocephaly, to distinguish it from congenital plagiocephaly.

Postural molding of the newborn's skull is common, and this presents clinically an occipital flattening, referred to as acquired plagiocephaly (or brachycephaly). Although some mild asymmetrical molding of the infant's cranial vault is likely common as a result of back sleeping, some babies develop severe cranial deformities that should be corrected. These deformities are typically characterized by flattening of only one occiput. The ipsilateral ear is displaced forward. There is compensatory bulging of the contralateral occipital area, the ipsilateral high parietal vertex, the ipsilateral temporal area, and occasionally the ipsilateral forehead. Bioccipital flattening is less commonly seen. These are acquired cranial deformities, and should be distinguished from congenital cranial deformities that result from the premature closure of a cranial surture (i.e., craniosynostosis). The latter condition frequently requires craniofacial surgery in order to correct the cranial deformity.

Positional plagiocephaly (postural molding of the cranium) may be prevented by periodically repositioning (turning over) the infant's head during sleeping. The “turn-over” repositioning treatment is not difficult to accomplish. However, to be effective this technique requires careful monitoring of the baby, diligence and the close attention of parents during sleeping hours. Although this seems simple in theory, in practice it is most difficult to accomplish consistently over the treatment term, which may extend up to 12 months, because of obligations parents may have to care for other children and attend to other matters, while at the same time trying to obtain the sleep and rest needed to carry on with work and other activities.

Infants more than three months of age and those who have not responded to repositioning may be treated with a custom-made cranial torque helmet. The torque helmet, which is precisely manufactured from an exact mold of the infant's head, continuously applies pressure or torque to the cranium to correct asymmetric deformities. The corrective forces have proven effective in some cases to restore cranial symmetry by helping the growing brain to reshape the cranium while it is still soft and compliant. The torque helmet is worn continuously, day and night, and is removed only for bathing until the child is twelve months of age or older. After twelve months of age or if the deformity is severe, torque helmets are of limited value and surgical cranial re-contouring may be required.

Custom-fitted, conventional torque devices have treated these acquired cranial deformities with varying degrees of success. The success has depended in large part on the age of the patient at the time torque treatment is begun. Clinical improvement occurs most rapidly in young infants (3 to 5 months of age). Treatment with these torque devices typically requires more time in older infants. As a child's age approaches 12 months, torque treatment becomes less effective. Many craniofacial physicians feel that little is gained with a cranial orthotic device after 12 months of age. Moreover, the acquired distortion of the base of the skull, as evidenced by the forward displacement of the ear on the side of the occipital flattening, does not generally improve with torque treatment devices. The petrous pyramids of the base of the skull tend to rigidly reinforce the skull base and resist external torsion/correction of the acquired cranial deformity.

Not infrequently, infants undergoing cranial torque treatment require re-fitting and replacement of the cranial orthosis to accommodate head growth as the child develops and the cranial deformity changes (responds). Because each orthosis is custom manufactured from an exact mold of the child's head, and because each device requires follow-up and modification as the child grows and the deformity responds, these devices are expensive and beyond the reach of many families, in particular those without effective insurance coverage. Some commercial insurance companies do not reimburse for the manufacture and use of such cranial orthotic devices, because the cranial deformities are acquired and are not the result of craniosynostosis (suture fusion).

It is therefore evident that a protective appliance is very much needed for all newborns and infants, in order to prevent the development of occipital flattening as a result of postural molding. Moreover, such a protective appliance should be universally available to all infants without requiring costly procedures to custom-fit the device to the individual infant. Rather, the protective appliance should be available on an “off-the-shelf” basis, using simple measurements such as head circumference to determine appropriate sizing. Finally, the protective appliance should be safe, simple to understand and use, relatively inexpensive and easily within the means of all families, even those without insurance coverage, so that preventive care and treatment can begin immediately after birth and continue at home without professional assistance other than the usual well baby check-ups.

Even though the “Back-to-Sleep” campaign by AAP has been successful in significantly reducing the number of SID incidences, there is still a risk of SID. And, some children will still roll over during the night at some point, even though most of the night may have been slept on the back. An infant can turn at any time during the night. It is not practical, nor feasible for a typical parent to continuously watch for an infant to roll over during the entire night. It only takes a few minutes for SID to occur. This presents a need for a better way of monitoring an infant's condition while sleeping, even if the infant spends most of his/her time sleeping on the back. Although most hospitals have expensive monitoring systems, there are very few systems that are practical and affordable for home use by consumers. Hence, there is a need for a system that monitors an infant's condition while sleeping, which is adapted for daily use by consumers at home and which is practical and affordable for home use.

### BRIEF SUMMARY

In one embodiment of the present invention, a protective cranial orthosis or some other similar head gear (preventive, corrective, or passive) includes at least one sensor for detecting at least one condition of the person wearing the head gear (e.g., infants). This head gear including one or more sensors may be part of a system that includes first and second base receivers for providing remote alarm emissions when one or more sensed conditions cross one or more threshold levels. Various types of sensors may be implemented into such system to measure conditions such as oxygen saturation level in the blood, pulse, and/or temperature, for example.

The protective appliance of an embodiment may be a cranial orthosis that is positioned around the head of a newborn or infant under one year of age, providing a protective shell that overlaps the occiput (os occipitale), left and right temporals (os temporale) and left and right parietals (os parietale). The protective shell has a concave profile with bilateral symmetry, and its interior surface is smoothly contoured to conform to the curvature and symmetry of the underlying occiput, temporal and parietal areas of the baby's head. Positional plagiocephaly (postural molding of the cranium) is prevented by redirecting the head weight forces that would otherwise compress the soft, compliant areas of the baby's head against the sleep surface and spreading those forces substantially uniformly over the smooth, conforming interior surface of the protective shell. The compressive forces imposed by the sleep surface (e.g., a mattress) are decoupled from the soft, vulnerable areas of the baby's head and are reacted through the protective shell. This prevents the development of a deformity and allows the developing areas of the infant's head to expand freely into the smooth, contoured cavity of the protective shell and thereby obtain normal cranial symmetry during the critical first twelve months of cranial development.

The concave pocket or cavity is sized to provide a close fit, to redistribute the compressive forces of the mattress over a large surface area of the baby's cranial vault. In the preferred embodiment, the protective appliance is in the form of a concave shell made of a durable, lightweight plastic material, having a head receiving pocket bounded by a smooth interior surface that is contoured to match the complex curvature and symmetry of the occipital, parietal and temporal regions of a normal human infant of the same age and gender.

The nominal dimensions (i.e., fronto-occipital circumference) and surface curvatures that characterize the cranium of a normal human infant are well known and documented in pediatric practice. It is also well known and universally recognized that the fronto-occipital circumference measurement (forehead to occiput) in a healthy human infant varies predictably in the population according to the infant's age and gender. Thus the protective appliance of an embodiment can be provided in standard, universal sizes (e.g., small, medium and large) and fitted effectively according to the age, gender and fronto-occipital circumference measurement of the infant as determined by traditional pediatric procedures.

In one embodiment, the protective appliance includes a crown portion, left and right wing portions and rostral end portions. The appliance is sized to cover substantially all of the underlying occipital area. The left and right wing portions extend bilaterally from the crown portion, overlapping the left and right parietal and the left and right temporal bones. Preferably, the upper parietal and frontal regions are only partially covered by the appliance in the protective position, thus allowing good air circulation and heat transfer over most of the infant's head, while protecting the compliant occiput from focused deformation forces applied by the sleep surface.

The wing portions are terminated by rostral end portions that are spaced apart and overlap the forehead (os frontale) area. The appliance is placed on the infant's head by spreading the rostral end portions slightly and inserting the baby's head into the protective pocket, and then allowing the rostral end portions to return to their resting (un-spread) position. Because the cranium is wider across the occiput than it is across the forehead, the appliance will be retained in the protective position by the rostral end portions, which yieldably oppose separation from the relaxed, protective position. The appliance includes a stretch band of soft woven fabric material, bridging the rostral ends of the appliance across the forehead region (os frontale) in order to help stabilize the appliance in the protective position.

In one embodiment, multiple layers of soft, spongy material or fabric material cover the contoured interior surface of the protective shell. The layers can easily be peeled away and removed at intervals to allow the appliance to accommodate normal head growth.

### BRIEF DESCRIPTION OF THE DRAWINGS

The accompanying drawing figures are incorporated into and form a part of the specification to illustrate the preferred embodiments of the present invention. Various advantages and features of the invention will be understood from the following detailed description taken with reference to the drawing figures in which:

FIG. 1 is a top plan view of one embodiment of the cranial orthosis fitted over the head of an infant in the protective position;

FIG. 2 is a left side elevation thereof, the right side elevation being the mirror image thereof;

FIG. 3 is a front elevation view thereof;

FIG. 4 is a front elevation view thereof showing a stretch headband attached to the orthosis and bridging across the forehead of the infant;

FIG. 5 is a lateral view of a human infant skull at birth showing the bones that make up the cranium and indicating in phantom the operative protective position of the cranial orthosis of the present invention;

FIG. 6 is a simplified elevation view of an infant's unprotected head resting on a sleep surface in the supine position, illustrating occipital flattening that occurs as the result of forces imposed by the infant's head weight and the reaction forces imposed by the sleep surface acting to compress a relatively soft, compliant occiput;

FIG. 7 is a simplified elevation view of an infant's protected head resting on a sleep surface in the supine position, illustrating the operative position of the cranial orthosis as it shields the infant's occiput;

FIG. 8 is a view similar to FIG. 7 showing the infant's head in nesting engagement with cranial orthosis as it distributes the head forces uniformly over the conformed interior surface;

FIG. 9 is a perspective view, partially broken away, of the cranial orthosis with its conformed interior surface covered by multiple layers of soft material that can be removed independently and sequentially to accommodate head growth;

FIG. 10 is a perspective view of the cranial orthosis of the present invention;

FIG. 11 is a chart that illustrates tabulated average and two standard deviation values of fronto-occipital circumference measurements for infant boys in the population age group from birth to age 24 months;

FIG. 12 is a chart that illustrates tabulated average and two standard deviation values of fronto-occipital circumference measurements for infant girls in the population age group from birth to age 24 months;

FIG. 13 is a perspective view of a flexible measuring tape used for determination of fronto-occipital circumference measurement;

FIG. 14 is a side elevation view of the tape being applied in a fronto-occipital circumference measurement;

FIG. 15 is a top plan view of a color chart used as a reference for comparison with colored lining layers;

FIG. 16 is a front elevation view of an embodiment of the present invention;

FIG. 17 is a bottom view of the sensor of the embodiment of FIG. 16;

FIG. 18 is a front elevation view of the sensor of the embodiment of FIGS. 16-17;

FIG. 19 is a partially cut away perspective view of the sensor of the embodiment of FIGS. 16-18;

FIG. 20 is a cut away top view of the sensor of the embodiment of FIGS. 16-19;

FIG. 21 is a system schematic of the embodiment of FIGS. 16-20;

FIG. 22 is a top plan view of an embodiment of the present invention;

FIG. 23 is a left side elevation view of the embodiment of FIG. 22;

FIG. 24 is another top plan view of the embodiment of FIGS. 22-23;

FIG. 25 is a perspective view of another embodiment of the present invention; and

FIG. 26 is a perspective view of yet another embodiment of the present invention.

### DETAILED DESCRIPTION OF THE INVENTION

The specification which follows describes a cranial orthosis intended for use by newborns and infants less than one year of age that will prevent the development of postural cranial deformities as a result of the child's sleeping on his or her back. Preferred embodiments of the invention will now be described with reference to various examples of how the invention can best be made and used. Like reference numerals are used throughout the description and several views of the drawing figures to indicate like or corresponding parts.

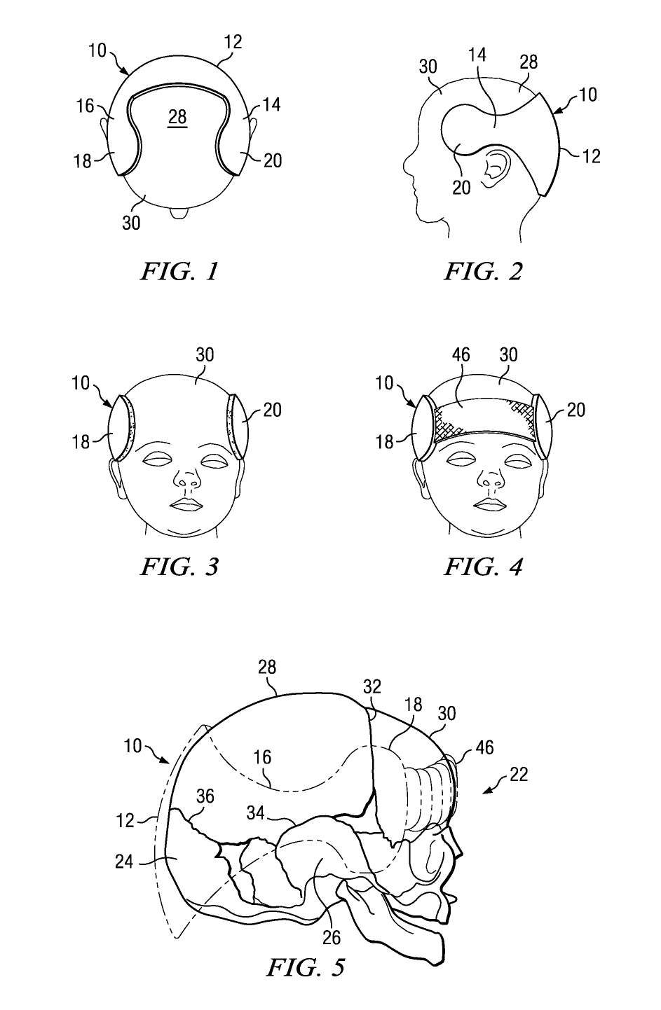

Referring to FIG. 1, FIG. 2, FIG. 3 and FIG. 10, the cranial orthosis of the present invention is in the form of a molded plastic appliance 10, for example a shell, headband or helmet, made of a unitary plastic molding or shell for protecting the soft, compliant skull base, occiput, left and right parietal bones and left and right temporal bones from deformation as the result of compressive forces caused by head weight while the infant is sleeping in the supine (face up) position on a sleep surface, for example a mattress. The protective appliance includes a crown portion 12 covering the left and right occipital areas, left and right wing portions 14 and 16 partially overlap the parietal, temporal and frontal areas. Rostral portions 18, 20 partially overlap the infant's forehead and help hold the appliance 10 in the operative protective position.

The crown portion 12 is centrally disposed for substantially complete overlapping coverage of the left and right sides of the occipital bone. The left and right wing portions 16, 18 extend bilaterally from the crown portion and the rostral end portions 18, 20 for terminal end portions on the wings. Preferably, the wing portions 14, 16 and rostral portions 18, 20 are dimensioned to provide limited overlapping coverage, whereby the upper parietal aspects of the bones 28, temporal bones 26 and frontal area 30 are only partially overlapped by the appliance in the protective position, thus allowing good air circulation and heat transfer over most of the infant's head, while shielding the soft, compliant occiput from direct contact against the sleep surface.

The protective, overlapping positions of the various protective elements of the appliance 10 can best be understood with reference to FIG. 5 that shows a cranium 22 of a normal human infant. The infant cranium includes an occipital bone area 24, a temporal bone area 26, parietal bone area 28 and frontal bone area 30 that encase the brain. These bones are separated by membranous intervals 32, 34 and 36 for several months and open cranial sutures until brain growth is complete, typically until teenage years. For the first year of life, an infant's skull is soft and pliable and can be deformed or flattened by the head weight of the infant as a result of the child's sleeping on his or her back.

This flattening deformity F, sometimes referred to as the “bean bag” effect, is shown in FIG. 6. Here, the soft occipital area 24 and temporal area 26 are compressed against the sleep surface 38 of a mattress 40. These soft, compliant areas deflect and are deformed inwardly along the line F, while the ipsilateral ear (and that side of the skull base) is displaced forwardly, with compensatory bulging of the contralateral occiput, the ipsilateral high parietal vertex and the ipsilateral frontal area.

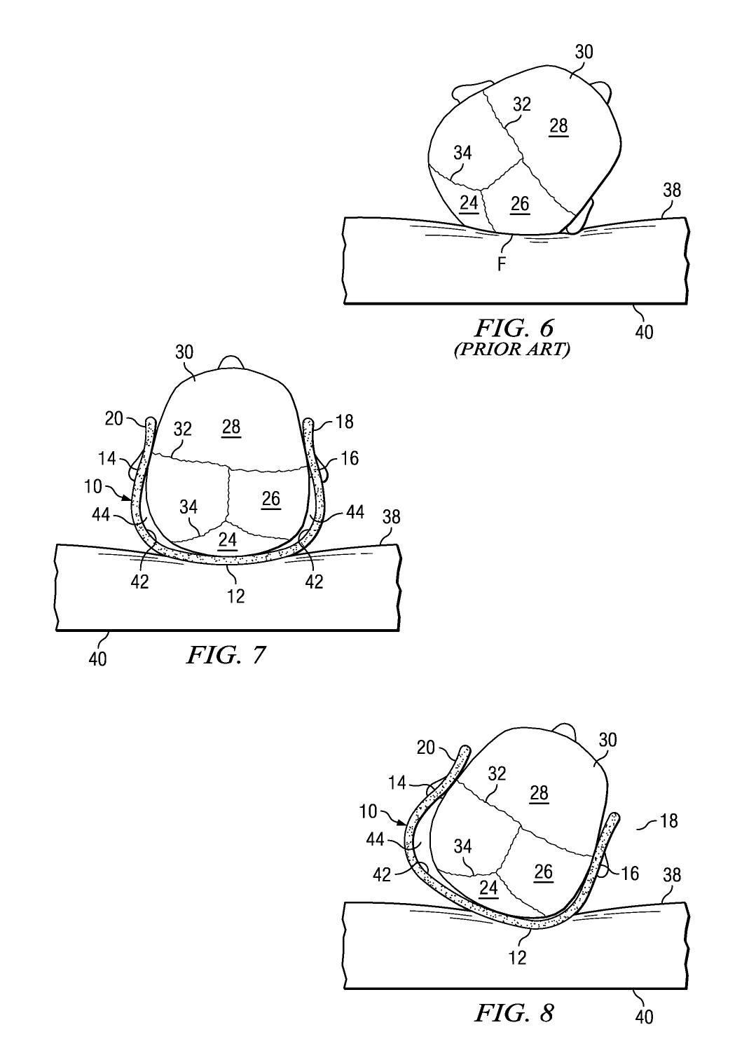

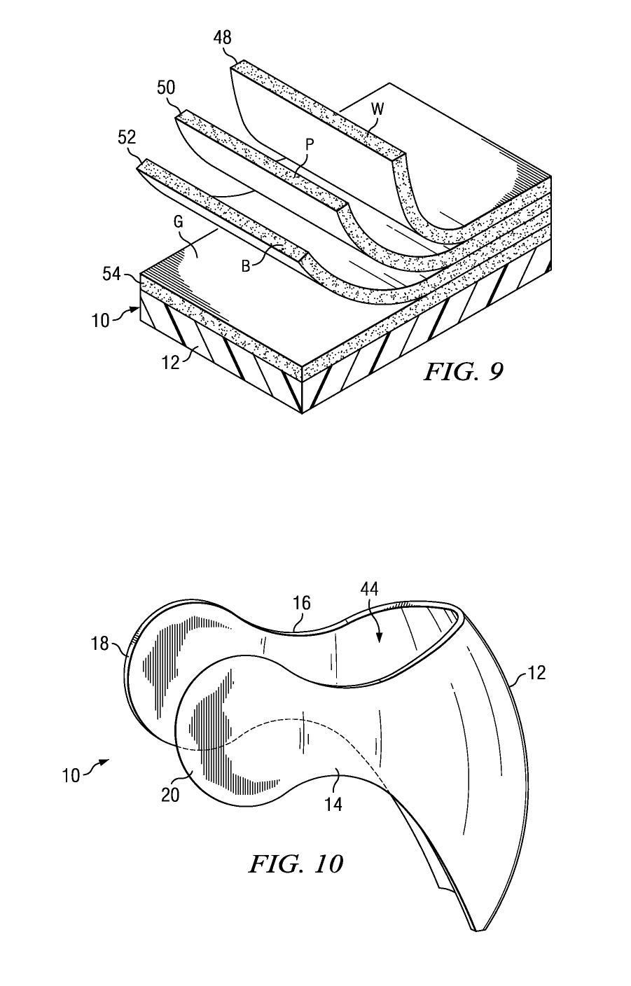

This acquired postural deformity is prevented by the cranial orthosis 10 that includes an interior surface 42 that is conformed in shape to the surface curvature of a normal human infant cranium, thereby defining a cavity or pocket 44 for receiving the head of an infant having compliant, developing head areas to be protected. In one embodiment of the invention, the cavity 44 is sized to provide a close, non-compressive fit of the conformed interior surface 42 in facing relation to the soft developing head areas to be protected, as shown in FIG. 7 and FIG. 8.

According to another arrangement, the conformed surface 42 and protective pocket 44 are slightly oversized relative to the head of the infant, thereby providing a close but non-interfering fit of the orthosis 10 about the infant's head. In this embodiment, the contoured interior surface is positioned in facing relation to the soft developing head areas to be protected, thereby allowing the orthosis to be worn while the infant is resting on a sleep surface in a supine position substantially without focusing torque forces on one particular part of the infant's head. This arrangement allows the infant's head to turn from side-to-side without imposing binding engagement of the orthosis against the soft, developing head areas.

According to yet another arrangement, the protective pocket 44 is dimensioned to allow nesting engagement of the infant's head against the conformed interior surface 42, as shown in FIG. 8. According to this embodiment, when the infant's head is received in the protective pocket 44, the infant's head weight forces are distributed substantially uniformly across the conformed interior surface 42 that nests in engagement against one or more of the soft developing head areas while the infant is lying on a sleep surface in the supine resting position.

The orthosis 10 is placed on the infant's head by spreading the rostral end portions 18, 20 slightly and inserting the baby's head into the protective pocket 44, and then allowing the rostral end portions to return to their resting (un-spread) position. According to an optional embodiment as shown in FIG. 4, a stretch band 46 of soft flexible material may be connected to the rostral end portions 18, 20 and bridge across the forehead 30 of the infant when the infant's head is received in the protective pocket 44. The stretch band 46 is formed by a strip of soft, resilient material, for example woven 100% cotton fabric, broadcloth of 65% polyester and 35% cotton or open cell foam material, and is reinforced by elastic. Other materials that can be used include knitted goods, velvet-like goods, and water resistant and water-proof fabrics, such as GORE-TEX® brand fabric. The stretch band is preferred for stabilizing the slightly oversized cavity embodiment of the orthosis 10 in the protective position.

The stretch band is optional and usually is not needed because of the retaining action of the rostrals 18, 20. Because the cranium 22 is wider across the occiput than it is across the forehead, the orthosis 10 will be retained in the protective position by the rostral end portions. The rostral end portions are resilient and yieldably oppose separation, but are spreadable to allow insertion and will return automatically to the relaxed, protective position shown in FIG. 1-FIG. 3 upon release.

According to another aspect of the invention, multiple layers of soft, spongy material or fabric material 48, 50, 52 and 54 cover the contoured interior surface 42 of the protective shell 12, as illustrated in FIG. 9. With the exception of the innermost base layer 54 which is permanently bonded to shell 12, the remaining layers are releasably bonded to each other by contact adhesive that permits independent release and removal of the strips one at a time. By this arrangement, the remaining layers 48, 50 and 52 can easily be peeled away and removed sequentially to accommodate normal head growth. Thus, the protective pocket 44 can be enlarged to accommodate normal growth of the infant's head, usually without requiring early replacement of the cranial orthosis 10, at least during the first three or four months. Typically, only two orthoses may be required for most infants, to accommodate normal head growth up to 12 months of age. Premature birth infants may require three orthoses.

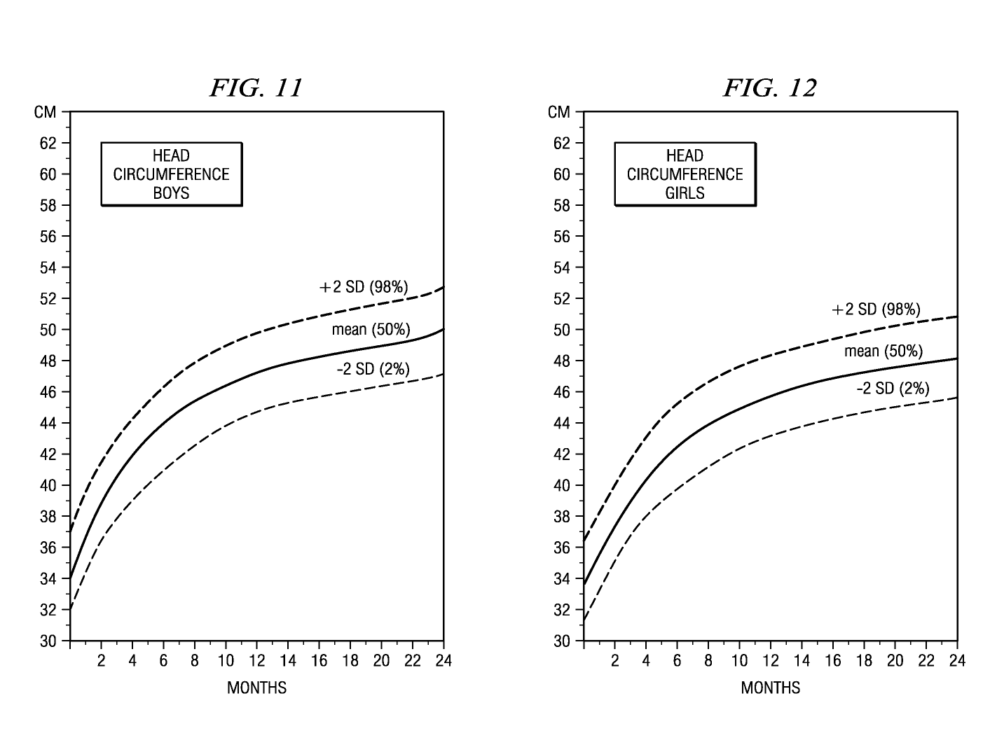

The protective shell 10 is molded with smooth interior surfaces that are contoured and conformed in shape to the surface curvatures of the occipital, temporal and parietal areas, respectively, of a human infant cranium having normal size, shape and symmetry of a healthy infant of a given age and gender. The nominal dimensions (i.e., fronto-occipital circumference) and surface curvatures that characterize the cranium of a normal human infant are well known and documented in pediatric practice. See, for example, the mean and standard deviation circumference values for boy infants shown in FIG. 11 and the mean and standard deviation circumference values for girl infants shown in FIG. 12, as tabulated by G. Nellhaus in Composite International and Interracial Graphs, Pediatrics 41: 106, 1968.

It is also well known and universally recognized that the fronto-occipital circumference measurement (forehead to occiput) in a healthy human infant varies predictably in the population according to the infant's age and gender, as shown in FIG. 11 and FIG. 12. For example, during the first eighteen months of age, the mean head circumference 22 increases from about 34 to about 48 cm for boys, and from about 34 to about 47 cm for girls. Thus the protective appliance 10 can be provided in standard, universal sizes (e.g., small, medium and large) and fitted effectively according to the age, gender and fronto-occipital circumference measurement of the infant as determined by traditional pediatric procedures.

According to the method of the invention, an inventory of protective appliances 10 is established, with each appliance having a pocket conforming substantially in size and shape to the cranium of a healthy human infant of given fronto-occipital circumference (FOC) measurement. The inventory includes protective appliances of various cavity sizes that may be indexed according to age, gender and average fronto-occipital circumference values tabulated for the general infant population.

Preferably, the inventory includes multiple cranial orthosis 10 in a range of cavity sizes that may be indexed according to age, gender and average fronto-occipital circumference values corresponding to male and female mean value circumference tabulations for the general infant population. For example, the standard sizes may range in maximum circumference from about 31 centimeters (corresponding to the 2nd percentile FOC of newborn females) to about 49.5 centimeters (corresponding to 98th percentile FOC of boys at twelve months), in four or six centimeter intervals. Three or four standard or universal sizes in six or four centimeter intervals, respectively, are sufficient to span the range from birth to twelve months for a given boy or girl. A closely conforming, non-binding initial fit is easily accomplished by selecting an oversized orthotic shell 10 and lining its conformed interior surface 42 with multiple release layers 48, 50 and 52. A satisfactory fit is maintained as the infant's head grows by removing one or more of the layers from time-to-time as discussed above.

The standard size protective appliances 10 are made from control prototypes fabricated from head molds of healthy control infants having normal head size, curvature and symmetry. A control infant's head should be symmetrically shaped and free of plagiocephaly. Head growth is monitored and a set of control molds are fabricated for each control infant to provide the 2-cm FOC size increments spanning the desired range, for example from 31 centimeters to 49 centimeters for an infant boy at the 50th percentile FOC. Optionally, an overall FOC span of 18 cm can be provided by a set of two control prototypes, from which two standard over-sized protective appliances 10 are fabricated, each fitted with four or five removable layers thereby providing adjustable fit in 2-cm FOC increments over an approximate range of 9 cm each (18 cm total per set), as described below.

Plastic molds are fabricated with reference to carefully selected control infants, and from these molds control prototypes are made in two or more standard or universal sizes. The standard size protective appliances 10 are then fabricated using the control prototypes as templates and using conventional mass production manufacturing techniques, for example by pneumatic thermoforming. In the preferred embodiment of the invention, the cranial orthosis is a shell molding 10 in the form of a head band fabricated of a light weight, high impact resistant plastic such as polypropylene, high density polyethylene, acetyl or polycarbonate resin having a sidewall thickness in the range of 1/16- 3/32 inch.

The age and gender of the infant are known, and the fronto-occipital circumference of the infant's head is measured. With this information, a protective appliance is selected from the inventory that most closely matches the infant's head size, age and gender, which accommodates normal head growth over a specified time period. Thus a physician can prescribe a protective cranial orthosis 10 from the established inventory of standard sizes based on the simple measurement of the infant's occipital-frontal circumference (FOC) measurement.

Exemplary materials for molding manufacture of the cranial orthosis 10 include engineering plastic materials such as ABS, polycarbonate, rigid polyvinyl chloride, polypropylene, acetyl, cellulose acetate butyrate, polystyrene or other high impact resistance plastic polymer resin material. For many applications more flexible plastic resins such as medium or low density polyethylene, plasticized polyvinyl chloride, polypropylene, ethylene vinyl acetate, butadiene styrene, vinyl acetate-ethylene or other suitable flexible plastic may be employed. Rigid or semi-rigid polyurethane, polyvinyl chloride, ethylene vinyl acetate, polyethylene or other suitable expandable plastic resins may also be utilized.

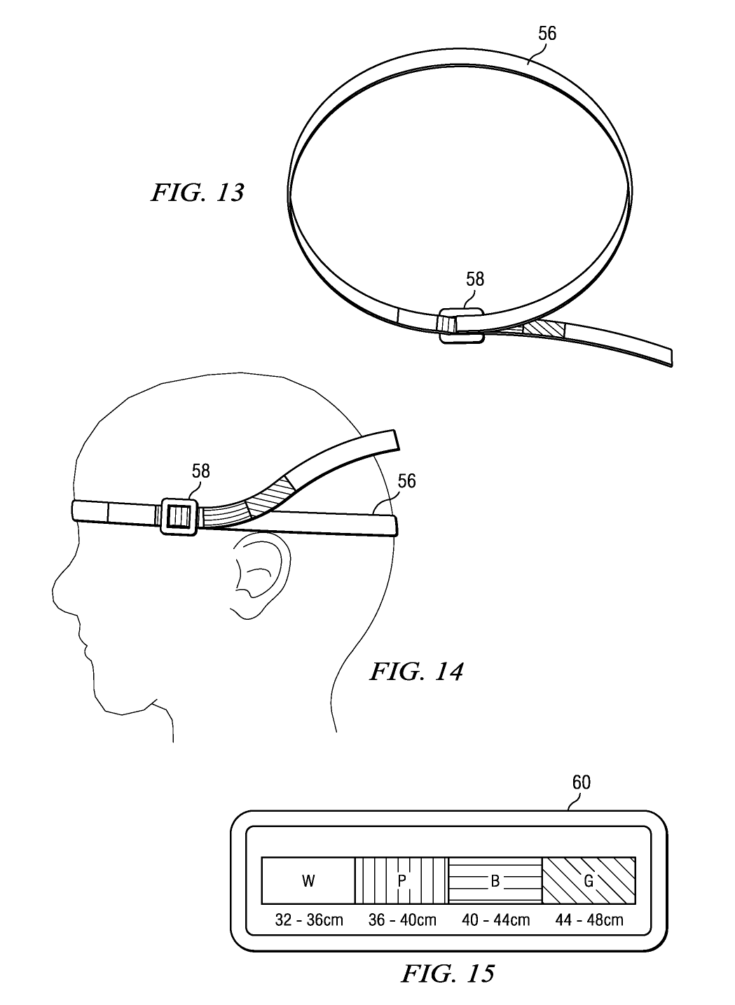

Referring now to FIG. 9, FIG. 13, FIG. 14 and FIG. 15, the removable layers 48, 50, 52 and 54 of soft fabric that cover the inner shell surface are provided in different colors, for example W (white), P (pink), B (blue) and G (green), to simplify the parents' understanding of when to remove a given layer to accommodate head growth. A flexible measuring tape 56 is fitted in a loop about the infant's head, as shown in figure and FIG. 15, with the loop closure position of the tape buckle 58 determining the FOC. The FOC measurement is indexed with reference to colored zones on the tape, for example W (white), P (pink), B (blue) and G (green). Preferably, each colored tape segment is 2 cm in length, corresponding with the expected growth range over a predetermined interval. Alternatively, the FOC measurement is taken with reference to an external color chart 60 having color zones W, P, B and G that cross-reference the FOC increments with the colors of the various fabric lining layers. The tape measurement is taken at weekly intervals to monitor the FOC and thus determine when to remove the current lining layer. By this method the parent can easily determine the most appropriate (best fit) lining layer by matching the color indicated by the FOC tape measurement with the color of the outer-most lining layer.

It will now be appreciated that a protective cranial orthosis has been described that is capable of preventing postural plagiocephaly in infants, can be mass produced at a nominal cost per unit, and can be made universally available to all infants without requiring costly procedures to custom-fit the orthosis to the individual infant. The protective appliance 10 of the present invention can be stocked and made available on an “off-the-shelf” basis, using simple FOC head circumference measurements to select the appropriate orthosis size from an inventory of standard size appliances. Because of its simple design and construction, the protective appliance is safe, easy to understand and use, relatively inexpensive and easily within the means of all families, even those without insurance coverage, so that preventive care and treatment can begin immediately after birth and continue at home without professional assistance other than the usual well-baby check-ups. With such early treatment, disfiguring cranial deformities that are so costly to treat and sometimes impossible to correct can easily be prevented by the cranial orthosis of the present invention.

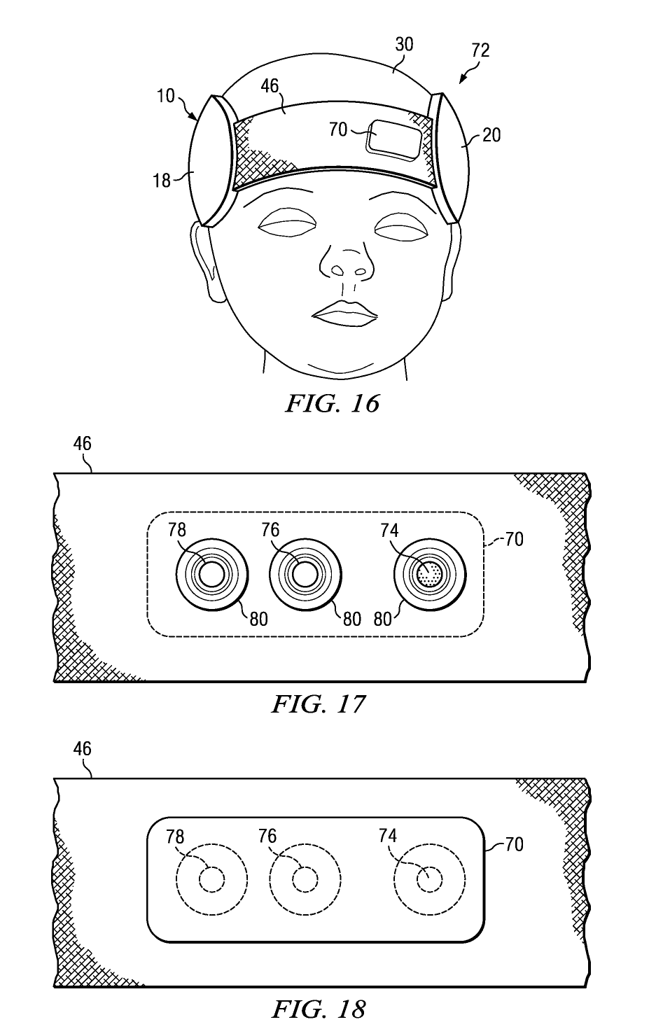

Next, embodiments will be described that combine a protective cranial orthosis 10 or some other similar head gear with at least one sensor 70 for detecting a condition of the infant (or any age person). It should be appreciated that although the sensor 70 is being presented in combination with a protective cranial orthosis or some other similar head gear, the sensor 70 may be used with a wrist band, waist band, ankle band or even another protective device, such as an arm or foot brace or cast. FIGS. 16-21 illustrate an embodiment that includes an oxygen saturation sensor 70, which is used to monitor the amount of oxygen in the infants blood.

In the event that an infant is suffocating, for example, the amount of oxygen in the infant's blood will drop. Because brain cells and organ tissues will die within minutes without proper oxygen levels, the measurement of the oxygen saturation level in the blood can provide a potentially life-saving alert that there is a problem, such as a SIDS-causing condition. Hospital grade systems for measuring oxygen saturation typically provide detailed data recordation, analysis, and display. The embodiment shown here is designed for home use, for example, and thus, the system 72 for measuring oxygen saturation may be greatly simplified to reduce the cost and make it easier to use. For an embodiment intended to prevent SIDS during daily home use, it may be sufficient to simply trigger an alarm or alert when oxygen levels sensed by the system fall below a certain or predetermined threshold level. For example, the threshold level and calibration may be set by the manufacturer, not being adjustable by the user to simplify the system and reduce its cost.

There are oxygen saturation sensors available on the market already which are designed for hospital use. For example, Somanetics Corporation provides a non-invasive oxygen saturation sensor that would work well in an embodiment of the present invention. U.S. Pat. Nos. 5,217,013, 5,584,296, and 5,902,235 (which are incorporated herein by reference for all purposes) owned by Somanetics Corporation describe an exemplary oxygen saturation sensor system. The Somanetics oxygen saturation sensor uses harmless near-infrared wavelength light to measure oxygen saturation levels in a person's blood. Light emitting diodes (LEDs) emit near-infrared wavelength light at the surface of the skin toward the brain. The sensor is preferably places on the temporal region of a person's forehead. Near-infrared light easily passes through scalp and bone tissue beneath the sensor. After the light is in vivo, it is either absorbed or scattered back up to the sensor. The sensor includes receivers for sensing both shallow and deep reflections of the light (depending on the wavelength of the light). Red-colored hemoglobin molecules within red blood cells have the highest light absorption of the near-infrared light emitted by the LEDs. The exact shade of red of each hemoglobin molecule indicates the amount of oxygen it is carrying. Thus, if the color of the hemoglobin changes beyond a threshold level during measurement, this can trigger an alarm or alert to indicate that there may be a sudden drop of oxygen in the blood (e.g., a possible SIDS situation).

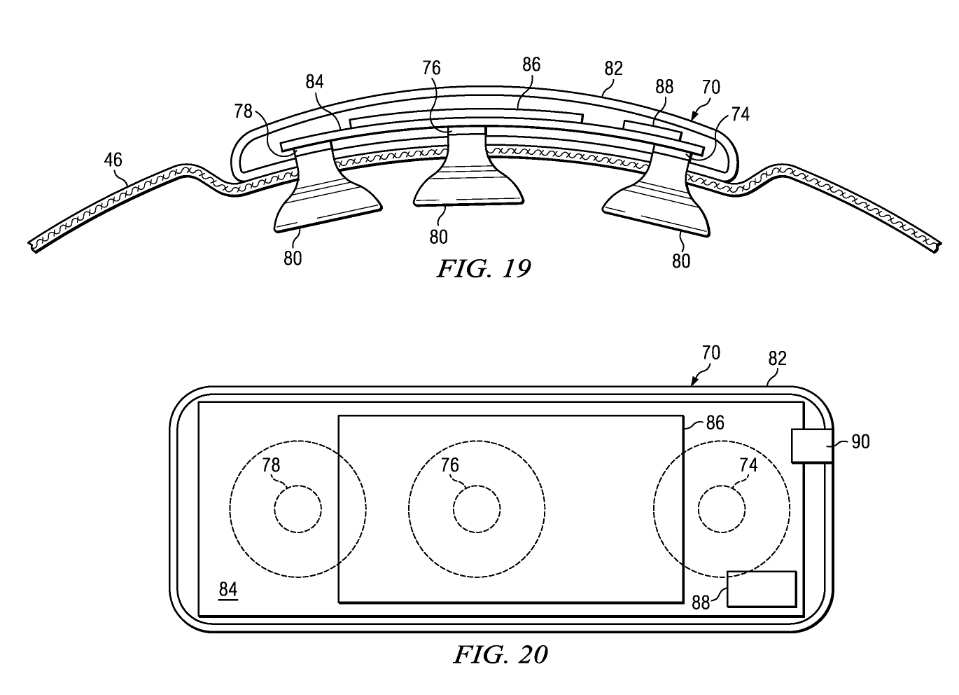

Referring now to FIGS. 16-21 in more detail, an oxygen saturation sensor 70 is located on the stretch band 46. FIG. 17 shows a bottom view of the sensor 70 located on the stretch band 46. This exemplary oxygen saturation sensor 70 has LEDs 74 for emitting various wavelengths of light (e.g., in the near-infrared and/or infrared ranges of light) for providing varying depths of light penetration. The oxygen saturation sensor 70 also has two receivers 76, 78, one adapted for receiving shallow field light reflections and one adapted for receiving deeper field light reflections. FIG. 18 shows a top view of the oxygen saturation sensor 70 located on the band 46. FIG. 19 shows a top perspective view of the oxygen saturation sensor 70 with portions of the sensor and band 46 cut away to show more details. In this example shown in FIG. 19, the sensor 70 has frustaconical-shaped cup portions 80 that are made from soft deformable elastic material (e.g., rubber, latex, or non-latex pliable material). This allows the sensor cups 80 to rest comfortably on the infants forehead for extended periods of time with less discomfort while also channeling the light transmissions to and from the sensor 70. In a preferred embodiment, the stretch band 46 has an elastic stretching force that is low so that the band rests gently on the infant's head while minimizing the force of the sensor 70 pressing on the infant's head.

FIG. 20 shows a top view of the oxygen saturation sensor 70 with part of the sensor casing 82 removed to expose components (in a simplified manner) within the sensor 70. The oxygen saturation sensor 70 of this embodiment includes a printed circuit board 84, a lithium-ion battery 86, a wireless transmitter 88 (e.g., Wi-Fi, Bluetooth, or other radio frequency transmission device), and connector 90. The connector 90 may be a standardized type of connector (e.g., Firewire, Mini Display Port, USB, USB2) for providing power to charge the battery 86, data retrieval, data uploads, software updates, or some combination thereof, for example.

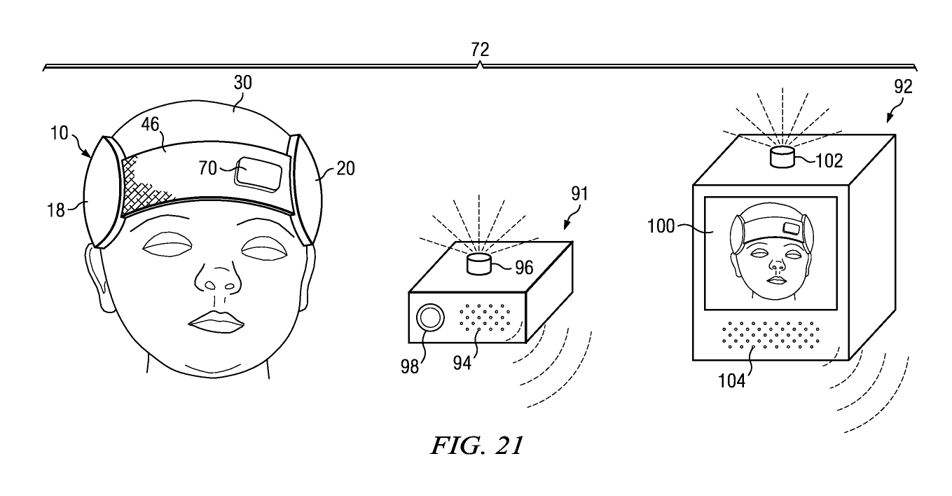

FIG. 21 shows the use of an embodiment of a protective cranial orthosis 10 that incorporates a sensor 70 for detecting a condition of the infant incorporated into a system 72. This exemplary system 72 includes a first base receiver 91 and a second base receiver 92. The first base receiver 91 includes a speaker 94 and a light 96 for providing audio and visual alarms in the event that the sensor 70 detects an unfavorable condition that crosses a threshold. In one embodiment, the first base receiver 91 is adapted for being positioned close to the infant so that the range of transmission required by the sensor 70 is reduced. This will provide numerous advantages, including (but not necessarily limited to) reducing the weight of the sensor 70 (smaller form, less pressure on infant's head), reducing the power needed by the sensor 70 (to extend battery life and allow for smaller battery 86), and reduced complexity of the sensor 70, for example. The first base receiver 91 is preferably plugged into a wall outlet for power supply. The first base receiver 91 may be used to provide an audible signal to awaken the baby via the speaker 94, which may allow the baby to shift position, cry, or other reactions that may avert SIDS. Arousability from sleep in response to a life-threatening event is a healthy, protective mechanism and one that is thought to be diminished in infants at risk of SIDS. Back-sleepers arouse from sleep more easily and sleep less deeply than tummy-sleepers. Thus, it is preferred that the audio alarm be capable of waking an infant from even a deep sleep, and preferably with an adjustable volume. Also, it may alert the parent or caretaker, if in the same room or in close enough proximity to hear the alarm. The first base receiver 91 includes a light 96 that can shine and/or flash when an alarm is triggered. This may provide a visual alert to the parent or caretaker, and/or it may help arouse or waken the infant. In one embodiment, the system 72 will allow the user to select what combination of alerts are provided at the first base receiver 91.

The first base receiver 91 of this embodiment of FIG. 21 also includes relay circuitry so that when an alarm signal is received from the sensor 70, the first base receiver can relay an alarm signal to the second base receiver 92. The system 72 may be designed so that the first base receiver 91 can transmit a signal to the second base receiver 92 at a greater distance (e.g., across the house) than the signal between the sensor 70 and the first base receiver 91. Hence, the second base receiver 92 may be located at a non-proximate location relative to the infant (e.g., in another room across the house). The first base receiver 91 also includes a camera so that the first base receiver 91 may be placed is visual proximity to the infant and then transmit a video image to the second base receiver 92. The first base receiver 91 also includes a microphone for providing an audio signal to the second base receiver 92. In another embodiment, the first base receiver 91 may be communicably coupled to a computer network (e.g., via ethernet wire, Wi-Fi, Bluetooth, Display Port, USB, USB2, etc.). Then, the relayed alarm signal from the first base receiver 91 may be sent to a second base receiver 92 or another computer device via a computer or telephonic network (e.g., Internet, WAN, LAN, VPN, Wi-Fi, etc.). Hence, the system 72 of an embodiment may be designed so that the second base receiver 92 may be at any distance away from the first base receiver 91.

In yet another embodiment, the first base receiver 91 may be formed by a port device plugged into a general purpose computer (e.g., PC, desktop, laptop, Macintosh) and the general purpose computer may have software executed thereon to provide processing of the signals from the sensor 70, triggering of alert signals (e.g., visible or audible alarms), and/or relay to a second base receiver 92 or to another computer device (e.g., another PC, desktop, laptop, smart phone, iPhone, iPod, MP3 player, television, set top box, home communication device, etc.) via a network connection (e.g., Internet, WAN, LAN, Wi-Fi, Bluetooth, VPN, etc.).

In one embodiment, the signal from the sensing of the infant's condition may be processed within the sensor 70 to determine whether to trigger or an alarm. Alternatively, in an embodiment, a raw signal or a signal with only minimal processing performed on it may be transmitted to the first base receiver 91. And in such case, the first base receiver 91 may have a processor and/or software algorithms (or firmware) that assesses the signal from the sensor 70 and makes a determination whether to trigger an alarm. An advantage of having the bulk of the processing performed by the first base receiver 91 rather than within the sensor 70 is that it may simplify the sensor 70, so that the sensor 70 requires less power to operate and is lighter in weight (less battery needed). Also, more advanced or more processor intensive algorithms may be run on the first base receiver 91. But advantages of having the processing performed within the sensor include the following: (1) many off-the-shelf sensors now include an ASIC and firmware for performing the analysis of the signal or conditioning the signal and are designed for low power operation (e.g., for cell phone and portable device applications); (2) the raw signal from the sensor may be analog and more complex to transmit than a processed signal; (3) processing of the signal may be performed faster at the sensor prior to any transmission of alarm status; and (4) by only sending a signal from the sensor 70 to the first base receiver 91 only when there is an alarm triggered (other than periodic handshakes to ensure that communication is still viable) may use less battery power than continuously transmitting data. With the benefit of this description, one may design a system that is optimize to minimize battery usage in the sensor 70 while still be reliable and while still being low in cost (e.g., using off-the-shelf components rather than custom built components).

Referring again to the exemplary embodiment of FIG. 21, the second base receiver 92 includes a video monitor 100, a visual alarm device 102 (e.g., LED, light), an audio alarm device 104 (e.g., speakers), and a wireless receiver device to receive the signal from the first base receiver 91 (or from a network connection). In an embodiment, the first and/or second base receiver may be a portable device (e.g., battery powered, belt clip casing). Because the second base receiver 92 can be used at any remote location that is not proximate to the infant (e.g., in another room of a house, or even outside while working in the yard or garden), a system embodiment of the present invention will provide freedom to the parent or caregiver to do other things while the infant sleeps, but with peace of mind that the infant is being monitored and while striving to prevent SIDS. With the benefit of this description, one may fashion a system using any combination of the elements mentioned for the first and second base receivers 91, 92, for example.

Any number of different sensors (alone or in combination) may be incorporated. In another embodiment of the present invention, the sensor 70 is a pulse sensor to detect whether the heartbeat rhythm has become irregular (e.g., in a SIDS-causing situation). There are many pulse sensors available that may be incorporated into an embodiment of the present invention. Some pulse sensors are piezoelectric pressure sensors that convert mechanical movement (i.e., expanding and contraction of a blood vessel as blood is pumped) to an electrical signal, which corresponds to the rhythm or rate of the heart beating. One of the downsides to these types of pulse sensors is the limited body locations on which the sensor may be placed and the physical contact requirements.

Blood vessel pulsation can also be measured using an optical sensor, which is a preferred means for an embodiment of the present invention. For example, the optical sensor 70 shown in FIGS. 16-21 may be used to measure pulse also or in alternative. Pulse oximeters capable of reading through motion induced noise are available from Masimo Corporation, for example. Moreover, portable and other pulse oximeters capable of reading through motion induced noise are disclosed in at least U.S. Pat. Nos. 6,770,028, 6,658,276, 6,157,850, 6,002,952, 5,769,785, and 5,758,644, for example, which are assigned to Masimo Corporation and are incorporated herein by reference for all purposes. Corresponding low noise sensors are also available from Masimo Corporation and are disclosed in at least U.S. Pat. Nos. 6,985,764, 6,813,511, 6,792,300, 6,256,523, 6,088,607, 5,782,757 and 5,638,818, which are assigned to Masimo and are incorporated herein by reference for all purposes. Such readings through motion pulse oximeters and low noise sensors have gained rapid acceptance in a wide variety of medical applications, including surgical wards, intensive care and neonatal units, general wards, home care, physical training, and virtually all types of monitoring scenarios.

In yet another embodiment of the present invention, the sensor 70 is a temperature sensor to measure the body temperature of the infant. Overheating, possibly by interfering with the central nervous system control of breathing, is another risk factor for SIDS. An infant lying on his/her back leaves the face and internal organs exposed so that they can radiate heat more readily than when sleeping on the stomach. An infant's prime avenue for heat loss is through his/her head and face, which more readily occurs when an infant is back sleeping. By monitoring the temperature of the infant while sleeping, increases of temperature can provide an indication that the baby may have rolled over or has his/her face buried in a sheet or pillow. Thus, when temperature measured at the temporal region rises above a predetermined threshold level, this can be an indication that a potentially dangerous situation or body position is existing. Triggering an alarm or alert at this time can provide a parent or caregiver notice of the situation, so the baby can be checked upon immediately.

Temporal temperature scanners measure the blood temperature in the temporal arteries of the forehead using infrared light. The arteries that carry blood directly from the heart provide the best assessment of true body temperature. The temporal arteries are ideal for accurate temperature measurements because they are located in close proximity to the heart and are readily accessible, lying just a millimeter below the skin surface of the lateral forehead region. Also, because the temporal arteries are highly profused and have very little basal motor activity, a steady flow of blood to its terminal forks is assured to this region, which always for accurate temperature measurements in most situations. Thus, an optical temperature sensor may be incorporated into the sensor 70 of an embodiment of FIGS. 16-21, in addition to or in alternative to other types of sensors, for example.

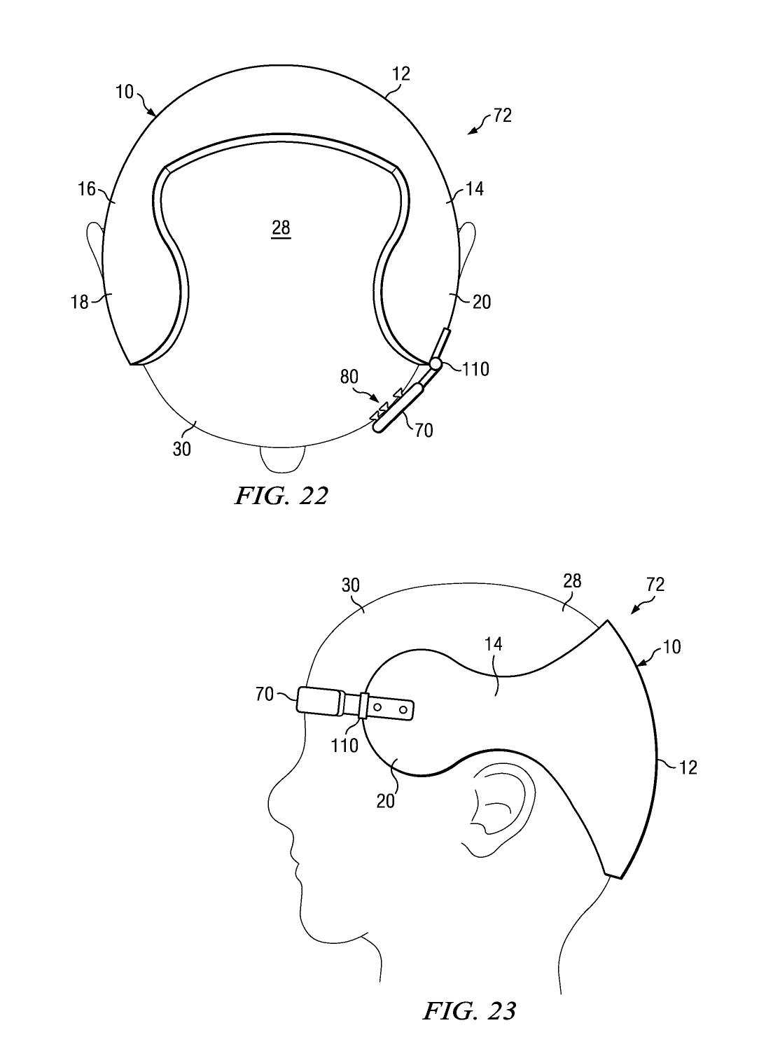

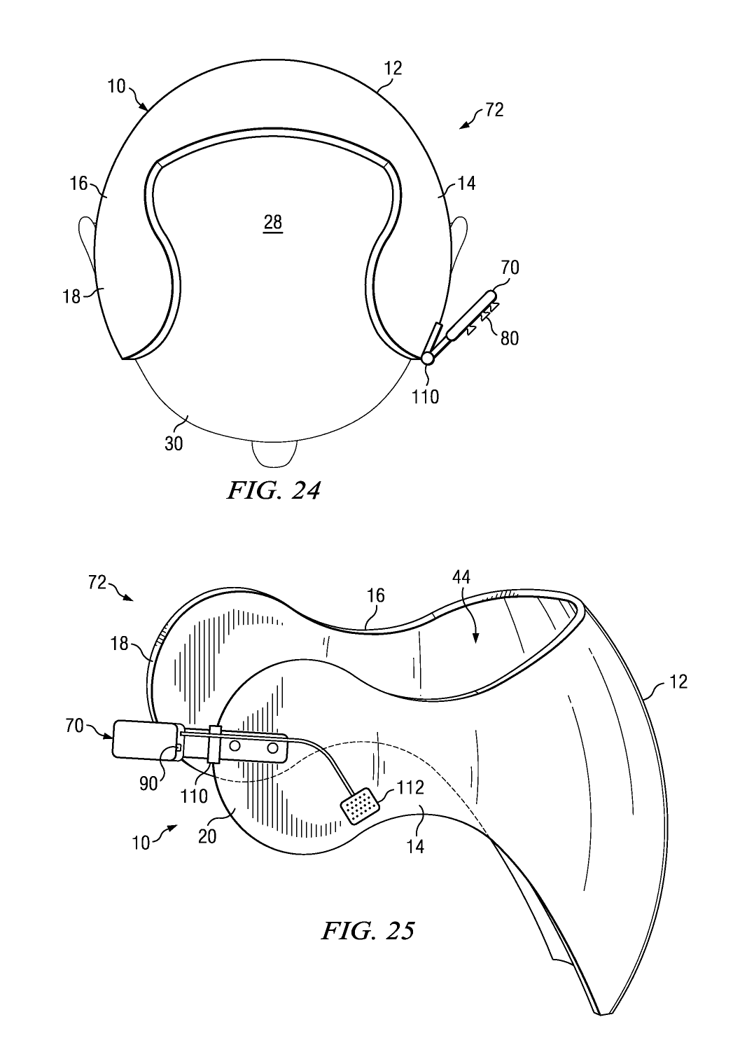

FIGS. 22-24 show another embodiment of the present invention. In this embodiment, the sensor 70 is attached to the protective cranial orthosis 10 by a spring biased hinge mechanism 110. The spring biased hinge mechanism 110 may gently bias the sensor 70 onto the forehead of the infant with enough pressure to keep it seated on the infant's head yet light enough that it does cause discomfort for the infant nor unneeded pressure on the infant's forehead (so that it does not interfere with the growth of the infant's skull). As with the embodiments of FIGS. 16-21, this embodiment preferably includes soft elastic frustaconical cup portions 80 to allow the sensor to rest well and to provide a channel for projected and reflected light, for example. As shown in FIG. 24, the hinge mechanism 110 will allow the sensor 70 to be positioned away from the infant's forehead at times (e.g., when applying lotion or when use of the sensor is not desired). An advantage of the sensor 70 being mounted via a hinge mechanism 110 is that it may be retrofitted to an existing protective cranial orthosis 10 or other head gear that previous was not equipped with a sensor 70. Also, the hinge mechanism 110 of an embodiment may be permanently attached or removably attached. Another advantage of this embodiment is that it does not require the use of the stretch band portion 46 (see e.g., FIGS. 16-21), which may be preferred by some people.

An embodiment of the present invention may also incorporate an alarm device 112 within or on the protective cranial orthosis 10. FIG. 25 shows an exemplary embodiment that includes an alarm device 112 on the protective cranial orthosis 10. The alarm device 112 may provide an alarm or stimulation that is audible, visual, motion, or combinations thereof, for example. Although the alarm device 112 in figure is shown as being separate from the sensor 70, in other embodiments, the alarm device 112 may be incorporated into the sensor 70 and/or into the sensor housing 82. An audible alarm may include a speaker that emits a sound to awaken the infant and/or to alert a parent/caregiver that is nearby. A visual alarm may include LEDs or other light emitting devices to awaken the infant and/or to alert a parent/caregiver that is nearby. A motion alarm may include a vibrator device, a motor, or other electromotive device, for example, that can provide some type of motion stimulation to the infant. This may be used to arouse or awaken the infant, which may help prevent a SIDS-causing event from continuing.

In an embodiment where an alarm device 112 is incorporated into or on the protective cranial orthosis 10, the system 72 may operate with the protective cranial orthosis 10 alone or in conjunction with one or base units (e.g., units 91 and/or 92 of FIG. 21). In a case where the system 72 is self contained and operates without a base unit, the sensor 70 may be simplified by not having a wireless transmitter device, for example.

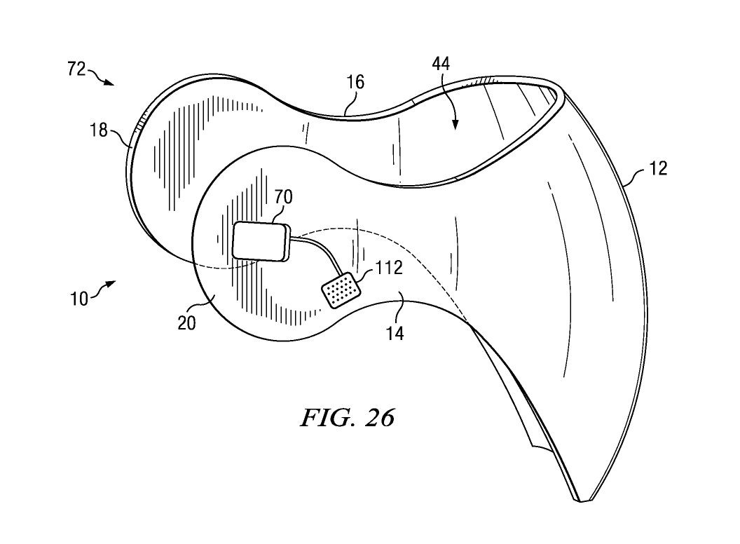

In another embodiment of the present invention, the sensor 70 is a motion sensor that detects, measures, and/or monitors the movement of the infant. FIG. 26 shows an exemplary embodiment where a motion sensor 70 is attached to the protective cranial orthosis 10. In this embodiment, the sensor includes a microelectromechanical system (MEMS), such as a gyroscope and accelerometer. MEMS devices are available in many different forms, sizes, and sensitivity levels. For example, STMicroelectronics supplies a large number of different MEMS sensors that are extremely small (a few millimeters in package size) and that only require small amounts of power to operate. Most such MEMS devices include an ASIC for processing the MEMS signal and outputting an analog or digital signal. Using a MEMS device with high sensitivity, the movement of the infant can be monitored and an alarm can be triggered if the infant goes without movement for a predetermined period of time, for example. Today's MEMS sensors are so sensitive, yet very small using semiconductor processing technology, that it could detect the rhythmic movement of the infant breathing. Thus, a properly designed/selected MEMS sensor could detect a lack of movement or lack of breathing, which can be an indicator of a SIDS-causing condition. As with the other embodiments described above, an embodiment where the sensor 70 is a MEMS motion detector device may include any of the aspects described in the other embodiments above, separately or in combination. For example, in the illustrative embodiment of FIG. 26, the sensor 70 includes one or more MEMS devices, a battery, a printed circuit board, a connector (for charging the battery), and a wireless transmitter.

An embodiment of the present invention may incorporate other types of sensors as well, separately or in conjunction with the types of sensors described above, for example. A sensor of an embodiment may include (but is not necessarily limited to) a blood pressure sensor, a mood sensor, microphone (preferably MEMS type), and other body function sensors, for example. An embodiment may incorporate a recording device to record data acquired by the sensor(s). Such recording device may be included in the sensor 70, mounted on/in the protective cranial orthosis 10, located at a base receiver 91/92 (e.g., signal transmitted by wireless or wired communication means), located in a general purpose computer, or some combination thereof, for example. The recording device may be analog, but is preferably digital using some type of memory device (e.g., EEPROM, flash, RAM, SRAM, DRAM, FRAM, magnetic disc, HDD, etc.).

Although it is preferred that an embodiment have its threshold limit(s) predetermined and set by the manufacturer (not by the user) for a home use system or portable system, the threshold(s) may be variable/adjustable by the user in an embodiment (e.g., via the connector, via a wireless communication interface with the sensor 70, via an upload, via a wire connection, etc.). Furthermore, it is contemplated that a sensor's alarm threshold may be automatically adjusted (e.g., via lookup table or software algorithm) based on ambient conditions (e.g., ambient temperature, ambient noise level, ambient light level, ambient vibrations, etc.) and/or based on a signal detected by another sensor in the system (e.g., increasing sensitivity or changing a threshold in one sensor if a certain threshold in another sensor is crossed), for example. An embodiment with multiple sensors can have the sensors dependent upon each other, and/or the alarm triggering may be dependent upon multiple sensors. Likewise, the sensor 70 in the protective cranial orthosis 10 may have its sensitivity or alarm threshold varied or adjusted via a base receiver signal (e.g., sensor in base unit or condition of base unit, such as proximity to the infant or ambient conditions). With the benefit of this disclosure, one can design a system to suit the needs, desires, and/or price point of a given market or application for an embodiment of the present invention.

Although the embodiments described above are wireless implementations, which is preferred to avoid wires that may be tangled or that may interfere with the infant, it is also contemplated that an embodiment may be wired; i.e., having a power and/or data signal wire extending from the sensor 70 to a base unit 91. In such case, the sensor 70 may be made lighter and smaller because it may not need a battery, a wireless transmitter, and other circuitry that may be placed in the base unit 91.

Although the embodiments described thus far herein have focused on the use or application of an embodiment of the present invention for an infant to prevent SIDS, for example, an embodiment of the present invention may also be used for any person of any age with the appropriate size adaption for the protective cranial orthosis, and for monitor other medical issues. For example, an embodiment may be used for an adult with sleep apnea or other sleeping disorders.

Although the embodiments described thus far herein have focused on embodiments using the protective cranial orthosis 10, it is contemplated here that an embodiment may be a sensor 70 combined with other types of preventative hear gear, corrective head gear, passive head gear (e.g., for keeping warm), or combinations thereof.

Although the invention has been described with reference to certain exemplary arrangements, it is to be understood that the forms of the invention shown and described are to be treated as preferred embodiments. Various changes, substitutions and modifications can be realized without departing from the spirit and scope of the invention as defined by the appended claims.

What is claimed is:

1. A cranial orthosis for preventing acquired plagiocephaly in infants having a soft developing head area to be protected, comprising:
a molded appliance having an interior surface that is conformed in shape to the surface curvature of a human infant cranium and operable to accommodate infant head growth;
two or more layers of soft, flexible material releasably disposed in overlapping nested relation and lining the conformed interior surface of the appliance thereby defining a protective pocket for receiving an infant's head, the protective pocket being sized to provide a close, non-compressive fit about the developing head area to be protected such that when an infant's head is received in the protective pocket and the infant is resting on a sleep surface in a supine position, the infant's head weight forces are spread substantially uniformly across the conformed interior surface facing the developing head area, whereby the lining layers can be removed one at a time to accommodate head growth; and
a sensor for detecting a condition of the infant, the sensor being coupled to the molded appliance for support.

2. The cranial orthosis for preventing positional plagiocephaly in infants as set forth in claim 1, wherein the sensor is an oxygen saturation sensor.

3. The cranial orthosis for preventing positional plagiocephaly in infants as set forth in claim 1, wherein the sensor is a pulse sensor.

4. The cranial orthosis for preventing positional plagiocephaly in infants as set forth in claim 1, wherein the sensor is a temperature sensor.

5. The cranial orthosis for preventing positional plagiocephaly in infants as set forth in claim 1, wherein the sensor is a motion sensor.

6. The cranial orthosis for preventing positional plagiocephaly in infants as set forth in claim 1, further comprising a wireless transmitter for sending signals generated by the sensor to a remote receiver.

7. The cranial orthosis for preventing positional plagiocephaly in infants as set forth in claim 1, wherein the sensor includes a battery and a printed circuit board.

8. The cranial orthosis for preventing positional plagiocephaly in infants as set forth in claim 1, further comprising a pliable headband portion extending between two portions of the molded appliance, wherein the sensor is attached to the headband portion.

9. The cranial orthosis for preventing positional plagiocephaly in infants as set forth in claim 1, wherein the sensor is attached to the molded appliance by a spring biases hinge mechanism.

10. The cranial orthosis for preventing positional plagiocephaly in infants as set forth in claim 1, further comprising an alarm device communicable coupled to the sensor.

11. The cranial orthosis for preventing positional plagiocephaly in infants as set forth in claim 10, wherein the alarm device is attached to the molded appliance.

12. The cranial orthosis for preventing positional plagiocephaly in infants as set forth in claim 10, wherein the alarm device includes a device selected from the group consisting of a speaker, a light emitting device, a vibrator, and an electric motor.

13. The cranial orthosis for preventing positional plagiocephaly in infants as set forth in claim 1, wherein the sensor is an oxygen saturation sensor, a pulse sensor, and an alarm device, wherein the alarm device is adapted to be triggered based on outputs of the oxygen saturation sensor and the pulse sensor.

14. The cranial orthosis for preventing positional plagiocephaly in infants as set forth in claim 1, wherein the sensor includes a recording device.

15. The cranial orthosis for preventing positional plagiocephaly in infants as set forth in claim 1, wherein the sensor is attached to an exterior surface of the molded appliance.

16. The cranial orthosis for preventing positional plagiocephaly in infants as set forth in claim 1, wherein the sensor includes a pliable, generally-cone-shaped portion adapted for providing a contact interface between the sensor and the infant during use of the sensor.

17. A system for detecting one or more conditions of a person, the comprising:
a preventative-condition headgear apparatus;
a sensor for detecting a condition of a wearer of the headgear apparatus, the sensor being attached to the headgear apparatus, wherein the sensor includes a battery, a printed circuit board, a wireless transmitter, and a wire connector; and
a first base receiver comprising circuitry for receiving signals from the sensor, wherein the first base receiver comprises an alarm device.

18. The system for detecting one or more conditions of a person as set forth in claim 17, further comprising a second base receiver comprising circuitry for receiving signals from the first base receiver, wherein the first base receiver includes a base unit transmitter for relaying alarms signals to the second base receiver based upon signals from the sensor.

19. The system for detecting one or more conditions of a person as set forth in claim 18:
wherein the first base receiver further includes a camera, a first light emitting alarm device, and a first sound emitting alarm device, and
wherein the second base receiver further includes a video monitor, a second light emitting alarm device, and second sound emitting alarm device.

20. A method of detecting one or more conditions of a person wearing a head gear apparatus:
sensing a condition of the person wearing the head gear apparatus, while the person is in a non-hospital environment, using a sensor that is physically coupled to the hear gear;
transmitting a signal from the sensor to a first base receiver; and
emitting an alarm if the signal indicates the one or more conditions of the person have crossed one or more selected threshold levels.
